(19) United States
(12) Patent Application Publication
(10) Pub. No.: US 2007/0238097 A1
(43) Pub. Date: Oct. 11, 2007

(54) METHOD USED TO SURVEY FOR PRESENCE OF AVIAN ANTIBODIES WHICH REFLECT EXPOSURE TO AN IMMUNOGEN

(76) Inventor: Hugh Fackrell, Windsor (CA)

Correspondence Address: MARK A KOCH, 866 MAIN STREET EAST, HAMILTON, ON, L8M1L9, US

(21) Appl. No.: 11/733,250

(22) Filed: Apr. 10, 2007

Related U.S. Application Data
(60) Provisional application No. 60/767,490, filed on Apr. 11, 2006.

(51) Int. Cl. C12Q 1/70; G01N 33/554; G01N 33/569; C12P 21/04

(52) U.S. Cl. 435/5; 435/7.32; 435/70.21

(57) ABSTRACT

A method to survey for activity of avian borne infections using an Avian Egg Vector Immuno Surveillance System. The method includes eggs collected from sentinel flocks in a desired area of surveillance. IgY, IgM and IgA antibodies are separated from the eggs. Separated antibodies are analyzed using an immuno assay such as ELISA for detection of avian borne infection. If positive, samples re-tested. If again positive, authorities informed. Authorities control vector causing spread of disease as necessary.

This application claims the benefit of previously regularly filed U.S. provisional application No. 60/767,490 filed on Apr. 11, 2006 by Hugh Fackrell under the title “Method Used To Survey For Presence of Avian Antibodies Which Reflect Exposure to an Immunogen”.

### FIELD OF THE INVENTION

The present invention relates to detection and surveillance of avian borne infections and more particular relates to an Avian Egg Vector Immuno Surveillance System used to survey for activity of avian borne infections.

### SUMMARY OF THE INVENTION

Traditional Methods used to detect and survey avian borne infections are discussed below. This patent application uses the West Nile virus as an example of an infection to which the present invention can be applied. The present invention however is applicable to any foreign molecule, immunogen or antigen introduced to an avian which stimulates the avian to produce an antibody. The vector by which the immunogen is transmitted in the case of West Nile is arthropods. There may be other types of vectors.

1. Bleeding Animals for Serum Antibodies

Current surveillance methods involve the testing of the blood of the sentinel birds for antibodies to West Nile virus (WNV) using a hemagglutination inhibition (HI) assay with confirmation by the MAC-ELISA. Throughout the arboviral season, blood samples are drawn every other week, and the NC Stat Laboratory of Public Health tests for the presence of antibodies for Eastern Equine Encephalitis (EEE), Highlands J (HJ), Saint Louis Encephalitis (SLE), and California Group (CAL) viruses. Due to cross reaction, the CAL reagent will also detect WNV. The disadvantages of this technique include its cost, a requirement for highly trained personnel, a high risk of exposure to live pathogen, decreased sensitivity due to stress on birds, time and personnel limitations, and only sentinel chicken flocks can be used.

2. Mosquito Indicators

North American mosquito species reported with WNV were tested using PCR technique. One disadvantage of this technique results from the eminent sensitivity to detect WNV nucleic acid in mosquitoes who may not have been able to transmit the virus, or may not have even contained live WNV. Therefore it is difficult to distinguish between past infection and the presence of live virus. A further disadvantage of this method is the inability to distinguish between infected individuals and infectious individuals.

3. Wild Bird Indicators

A dead wild bird index has been used to predict where and when human cases would appear. This does not appear to have been effective because WNV—associated mortality is not an accurate indicator of human risk or the spread of disease, and it does not confirm the presence of the virus.

Therefore there is need for a new and improved method to survey for activity of avian borne infections and the present invention proposes a new method which uses Avian eggs to survey a certain geographic area for the activity of avian born infections and one example of this is the West Nile Virus.

### BRIEF DESCRIPTION OF THE DRAWINGS

The invention will now be described by way of example only with reference to the following drawings in which:

FIG. 1 is a flow chart showing the steps of the method used to survey for activity of avian borne infections.

FIG. 2 is a flow chart showing the steps performed to isolate the antibodies IgY, IgM and IgA from chicken eggs.

FIG. 3 is a flow chart showing the steps of the method used to survey for activity of avian borne infections and showing the steps performed to isolate the antibodies IgY, IgM and IgA.

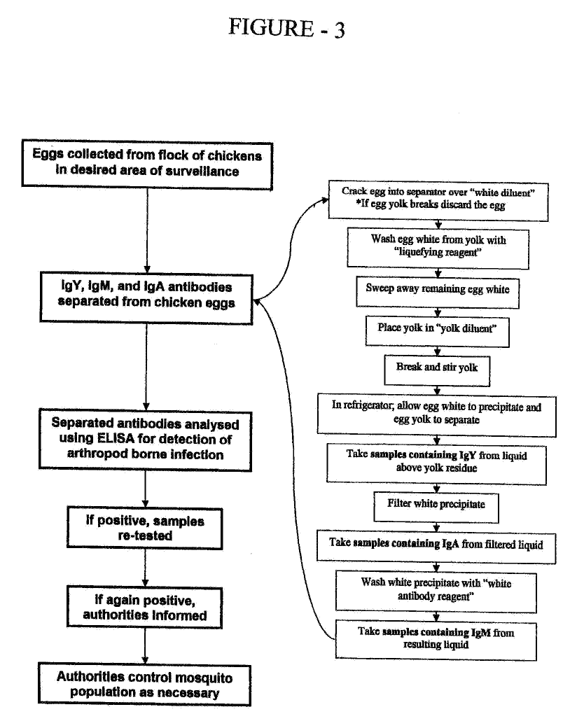

### DETAILED DESCRIPTION OF THE PREFERRED EMBODIMENT

Definitions

Antibodies—any of a large number of glycoproteins of high molecular weight that are produced normally by specialized immune cells after stimulation by an antigen in an immune response—called also immunoglobulin (Ig).

Arbovirus—any of various RNA viruses transmitted chiefly by arthropods.

Arthropod borne infection—viruses which could be transmitted to man via an insect.

Hemagglutination inhibition (HI) assay—a test in which the inhibition of red blood cell agglutination is a marker of the presence of specific antibodies in a solution.

IgG/IgM/IgA—different types of antibodies (immunoglobulins).

IgY—chicken IgG antibodies.

Enzyme-linked immunosorbent assay (ELISA)—a quantitative in vitro test for an antibody or antigen in which the test material is absorbed on a surface and exposed to a complex of an enzyme linked to an antibody specific for the substance being tested for with a positive result indicated by a treatment yielding a color in proportion to the amount of antigen or antibody in the test material (MAC-ELISA)—specific ELISA for IgM detection). ELISA is the favored method for testing the isolated antibodies there are other immuno assays available which produce useful results. For example it is possible to use Agar Gel Immuno Diffusion (AGID), or Fluorescent Immuno Assay (FIA). Each method has its own advantages and disadvantages well known in the art. In this patent references to use of ELISA is one example of an immuno assay test which can be used. ELISA may be substituted with other immuno assay tests.

Nucleic acid—any of various acids (as DNA or RNA) that are composed of nucleotide chains.

Polymerase chain reaction (PCR)—an in vitro technique for rapidly synthesizing large quantities of a given DNA segment; can be used to detect a specific DNA segment.

Egg Antibodies

A laying hen can produce a 50-60 gm egg (more antibodies than a typical blood sample) every 24 hours. Egg antibody titres directly reflect the hen's immune status. Chickens secrete all three classes of antibodies into their eggs. IgG (aka IgY), found in extremely high concentrations, only in the yolk has been exploited as a diagnostic reagent for 25 years. Antibodies in egg white, i.e. IgM and IgA, have been ignored.

The presence of IgY to any particular infection is not a predictor of infection activity in any given area.

Egg white, with its IgM antibodies is deposited in the forming egg over a period of 3 hours. IgG (yolk) is deposited in the forming egg over a period of weeks. IgG can only tell analysts that; a particular hen has had an infection in the past weeks. IgM will tell analysts that a particular hen is infected right now. The inventor, has isolated and measured the biological activity of all three classes of chicken egg antibodies. For the first time, eggs become an attractive, alternative, concentrated, source of avian antibodies for the screening of sentinel flocks.

Chicken eggs are an attractive, alternative to serum, concentrated source of avian antibodies. There is less risk of viral exposure to personnel as WNV virus is not transferred into eggs. Eggs can be obtained easily from sentinel or domestic flocks. Eggs may be transported safely and are stable for extended periods of time. Egg laying is much less stress on the bird than venipuncture and antibody yields are greater. The interfering IgG, found only in the yolk, is conveniently segregated from IgM and IgA. The latter two antibodies are enriched in the egg white. Egg white with its accompanying IgM antibodies is deposited over a very few hours. The presence of antibodies in eggs laid on any particular day mirrors the appearance of IgM in the serum the previous day.

IgY:

Viable in high salt concentrations but not in urea

Can be broken down by proteases

Not effective in the digestive system

Accounts for 80% of total serum antibodies

Readily crosses the placenta

Plays active role in fetal protection

Deposited in forming egg over a period of weeks

IgM:

Accounts for 5-10% of total serum antibodies

First antibody produced upon exposure to a pathogen

First antibody produced by the neonate

Indicates direct blood exposure to infection (arthropod vector)

Deposited in forming egg over a period of 3 hours

IgA:

Secretory antibody

Viable in high urea and high salt concentrations

Easily transported across membranes (may indicate secondary infection vector such as exposure through mucous membranes—unimportant to human infection)

Sentinell and Pre-Existing Flocks

Sentinell flocks provide a direct measure of the number of WNV transmitting mosquitoes in an area. Through antibody testing of flocks we can gauge the human risk according to the number of infected birds in the flock as compared to the non-infected birds. The essential component of sentinel chicken surveillance is location.

Pre-existing flocks refers to chicken farmers who happen to operate farms in the areas of surveillance. Since there is no need to bleed chickens there is need to handle the actual chickens. There need only be an arrangement made to purchase eggs directly from the farmer. The eggs would then be tested just as they would if they were from a placed sentinel flock.

Advantages To New Method

Egg Antibodies:

There is far less risk of active WNV to personnel as WNV is not transferred into eggs

Eggs are easily obtained, safely transported and are stable for extended periods.

Egg laying is less far stressful than bleeding

No skill is required to collect eggs as compared to serum collection

Egg collection is far cheaper than collecting serum

Eggs can be collected daily as opposed to 1-2 times per week at best for serum.

Sample size is larger for eggs than serum

There is less concern about animal rights activists opposed to the treatment of bled chickens.

Sentinel Flocks

Long history of successful use in arbovirus surveillance.

Mortality in birds is low

Birds are not reservoirs for West Nile Virus (WNV)

Geographic location of infection is clear

Collection of specimens (eggs) is inexpensive

A flexible system that can be expanded and contracted as needed and time and place of exposure are known.

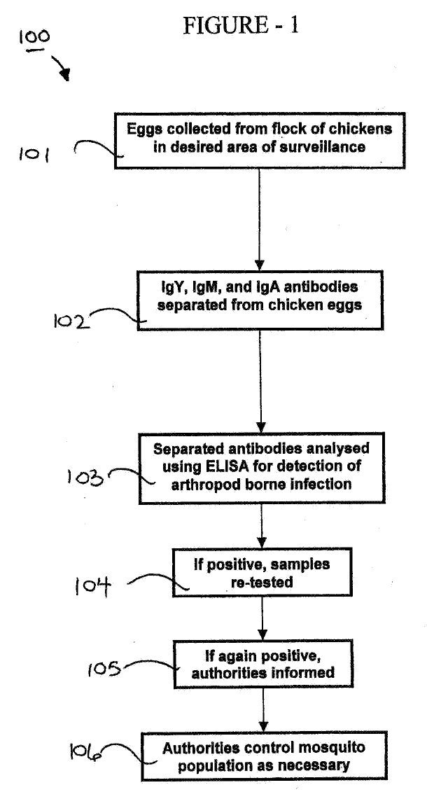

The present invention, a method used to survey for activity of avian borne infections is shown generally as 100 in FIG. 1. This new method would use Avian eggs to survey a certain geographic area for the activity of avian borne infections like West Nile Virus. This process is termed by the inventor as Avian Egg Vector Immuno Surveillance (AEVIS) system which involves the following steps which are shown in FIG. 1.

Step 1 shown generally as 101 involves collecting eggs from a flock of chickens in the desired area of surveillance.

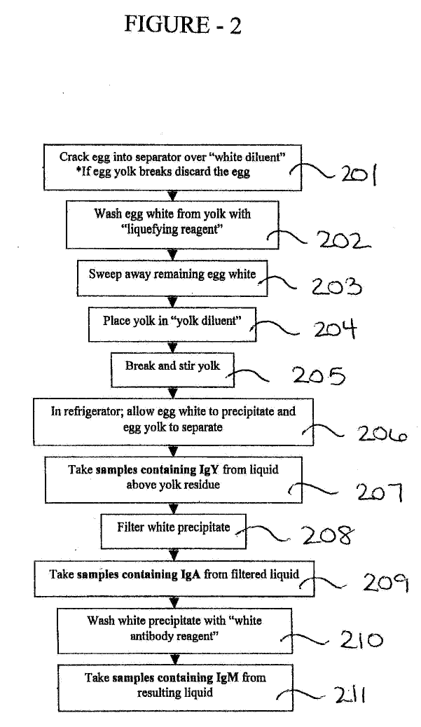

Step 2 shown as 102 in FIG. 1 includes, separation of the anti bodies IgY IgM and IgA from the chicken eggs. The method for separation of these antibodies is depicted in FIG. 2 and described more fully below.

Step 3 shown as 102 in FIG. 1, separated anti bodies are analyzed using an immuno-assay such as ELISA for detection of avian borne infections.

Step 4 shown as 104 in FIG. 1, in the event of a positive finding, samples are retested to confirm the test results.

Step 5 shown as 105 in FIG. 1, a second positive test will result in informing the authorities of the avian borne infections.

Step 6 shown as 106 in FIG. 1, the authorities will have enough information in hand in order to make the necessary judgments in regard the control of mosquito populations or control of another vector in the area in which avian borne infections are detected.

Chickens secrete all three classes of antibodies into their eggs; IgC (in chickens, called IgY) in the yolk, IgM and IgA in the egg white. IgY is a marker of an infection in the past weeks, while IgM is marker of current infection. The biological activity of all three classes has recently been measured, making eggs a new and acceptable source of avian antibodies. The technique of antibody separation is outlined in the following steps which are depicted in FIGS. 2 as follows:

The first step shown as 201 in FIG. 2 requires an egg to be cracked into separator over white diluent (if egg white breaks discard the egg).

Step 2 shown as 202 in FIG. 2, the egg white is washed from the yolk with liquefying reagent.

Step 3 shown as 203 in FIG. 2, remaining egg white is swept away.

Step 4 shown as 204 in FIG. 2, the egg yolk is placed in yolk diluent.

Step 5 shown as 205 in FIG. 2, the egg yolk is broken and stirred.

Step 6 shown as 206 in FIG. 2, upon refrigeration; allow egg white to precipitate and egg yolk to separate.

Step 7 shown as 207 in FIG. 2, take samples containing IgY from liquid above yolk residue.

Step 8 shown as 208 in FIG. 2, filter out the white precipitate.

Step 9 shown as 209 in FIG. 2, take samples containing IgA from the filtered liquid.

Step 10 shown as 210 in FIG. 2, wash white precipitate with white antibody reagent.

Step 11 shown as 211 in FIG. 2, take samples containing IgM antibody from resulting liquid.

There are many advantageous to using chicken egg antibodies over chicken blood. There is less risk of viral exposure to personnel using eggs and no skill is required to select the eggs. Eggs are easily and cheaply obtained, safely transported, stable for extended period of time and can be collected daily. Furthermore the sample size if larger for eggs than it is for serum. Lastly, there is less concern about animal rights and egging is far less stressful than bleeding.

There are also many advantageous to using the entire Avian Egg Vector Immuno Surveillance (AEVIS) system. There is a long history of successful use of sentinel flocks in arbovirus surveillance. The geographic location of infection is clear and the time and place of exposure are unknown. Furthermore, the system is expandable and can be contracted as needed.

This patent application uses the West Nile virus as one example only of an infection to which the present invention can be applied. The present invention however is applicable to any foreign molecule, immunogen or antigen introduced to an avian which stimulates the avian to produce an antibody. The vector by which the immunogen is transmitted in the case of West Nile is arthropods. There may be other types of vectors. The present invention is applicable for example to avian flu which is spread from bird to bird. The present invention can for example be used to detect the presence of avian flu at the farm using locally gathered eggs.

Whatever infects an avian can be detected in their eggs, such as for example salmonella, E. Coli, staphylococcus and lime disease. These are just some examples of infections which may be detected by the present invention.

What is claimed is:

1. A method to survey for activity of avian borne infections using an avian egg vector immuno surveillance system which includes the following steps:
a) collecting eggs from a sentinel flock in the desired area of surveillance;
b) separating antibodies from the eggs;
c) analyzing separated antibodies using an immuno assay test for the detection of avian borne infection;

2. The method claimed in claim 1 wherein step b including separating IgY, IgM and IgA antibodies from the eggs.

3. The method claimed in claim 1 wherein the immuno assay test including ELISA for detection of arthropod borne infection.

4. The method claimed in claim 1 wherein the sentinel flock including a flock of chickens.

5. The method claimed in claim 1 wherein the antibodies are separated from the eggs by including the steps of:
a) separating the white from the yolk;
b) using suitable diluent with egg white;
c) allowing egg white to precipitate;
d) collecting antibodies.

6. The method claimed in claim 5 wherein the antibodies are separated from the eggs by including the steps of:
a) filtering white precipitate;
b) collecting samples containing IgA antibody from filtered liquid.

7. The method claimed in claim 6 wherein the antibodies are separated from the eggs by including the steps of:
a) washing white precipitate with white antibody reagent;
b) collecting samples containing IgM antibody from resulting liquid.

8. The method claimed in claim 1 wherein the antibodies are separated from the eggs by including the steps of:
a) separating the white from the yolk;
b) using suitable diluent with yolk,
c) allowing yolk to separate;
d) collecting antibodies.

9. The method claimed in claim 8 wherein the antibodies are separated from the eggs by including the steps of:
a) collecting samples containing IgY antibody from liquid above the yolk residue.

10. A method to survey for activity of avian borne infections using an avian egg vector immuno surveillance system which includes the following steps:
(a) collecting eggs from a sentinel flock in the desired area of surveillance;
(b) separating IgY, IgM and IgA antibodies from the eggs;
(c) analyzing separated antibodies using ELISA immuno assay test for the detection of avian borne infection;
(d) retesting samples with positive results;

11. The method claimed in claim 10 wherein the antibodies are separated from the eggs by including the steps of:
a) separating the white from the yolk using a liquefying agent;
b) using suitable diluents with egg white and egg yolk;
c) allowing egg white to precipitate;
d) allowing egg yolk to separate;
e) collecting IgY antibodies from liquid above yolk residue;
f) collecting IgA antibodies from filtered egg white liquid;
g) collecting IgM antibodies from egg white precipitate.
